(19) United States
(12) Patent Application Publication
(10) Pub. No.: US 2009/0054964 A1
(43) Pub. Date: Feb. 26, 2009

(54) PACEMAKER ELECTRODE WITH HELICAL COIL AND FEED LINE COIL

(76) Inventor: Peter Osypka, Rheinfelden-Herten (DE)

Correspondence Address: VOLPE AND KOENIG, P.C., UNITED PLAZA, SUITE 1600, 30 SOUTH 17TH STREET, PHILADELPHIA, PA, 19103, US

(21) Appl. No.: 12/192,438

(22) Filed: Aug. 15, 2008

(30) Foreign Application Priority Data
Aug. 22, 2007 (DE) 102007039554.1

(51) Int. Cl. A61N 1/05

(52) U.S. Cl. 607/127

(57) ABSTRACT

A pacemaker electrode (1) with a helical coil (3) arranged in a metal housing (7) is provided. So that no undesired electrical voltage pulse can occur if there is mutual contact between the helical coil (3) and metal housing (7), a permanent electrical connection of the metal housing (7) is provided with the helical coil (3). This electrical connection is realized by an electrical conductor (8), which is advantageously constructed as an additional coil and which is connected directly or indirectly to a part, which is permanently connected in an electrically conductive way to the rotating plug pin (4) and the feed line coil (4) connected to this plug pin and leading to the helical coil (3) by a wiping contact (12).

### CROSS-REFERENCE TO RELATED APPLICATIONS

This application claims the benefit of German Application DE 10 2007 039 554.1, filed Aug. 22, 2007, which is incorporated herein by reference as if fully set forth.

### BACKGROUND

The invention relates to a pacemaker electrode with a helical coil, which is retracted during its implantation and which can be extended in the axial direction through rotation from the retracted position into an active holding position for engaging in the heart tissue and which is connected in an electrically conductive way via an electrically conductive feed line coil to a plug pin that can be introduced into a pacemaker, wherein the helical coil that can move in the axial direction into the position of use is arranged on the distal end of the pacemaker electrode and is held by a holder in a housing, which is made from metal and which is suitable or constructed as a cathode for measuring the stimulus threshold on the heart for a helical coil still located in the retracted position, and a long-lasting electrical connection of the metal housing to the helical coil of the pacemaker electrode is provided.

A comparable pacemaker electrode is known from U.S. Pat. No. 6,687,550 B1. The helical coil is here located within a metallic sleeve, which acts as a metal housing and which has a peripheral collar on its end face. Through an additional compression spring, a long-lasting electrical connection of the metallic housing to the helical coil is to be maintained. Here, additional built-in parts, which have corresponding space requirements, must be provided on the distal end of the pacemaker electrode, even though it is desirable that, above all, the distal end of such an electrode has a construction that is as small and space saving as possible.

### SUMMARY

Therefore, it would be desirable to provide a pacemaker electrode of the type noted above, in which contacts between the helical coil and the metal housing containing it do not interfere with the pacemaker and nevertheless the distal end has a construction that is as space saving as possible.

To meet this need, according to the present invention the pacemaker electrode defined above is provided such that the metal housing and/or a holder of the helical coil that is adjustable in the axial direction are connected to an electrical conductor, which leads along the feed coil to a proximal sleeve-shaped part or sleeve part, relative to which the feed line coil and/or a continuation part of the feed line coil can rotate, and that the fixed sleeve part and the rotating continuation part and/or the rotating feed line coil are connected in an electrically conductive way by a wiping contact. In this way it is achieved that the electrical connection of the metal housing to the helical coil is also maintained during its rotation. Thus, there is also not the risk that additional voltage or current pulses are generated due to additional contacts between the helical coil and the metal housing. By moving the long-lasting electrical connection to the proximal end of the pacemaker electrode, the distal end can also have a construction that is as space saving and as small as possible.

An effective construction can be developed when the plug pin or the continuation part connected to the plug pin is connected to the feed coil and when the wiping contact is provided on a non-rotating sleeve part arranged concentric thereto. Thus, a relative rotation of the feed line coil relative to the fixed sleeve can take place, whereby the helical coil is rotated and simultaneously adjusted in the axial direction, wherein simultaneously the wiping contact becomes effective and provides for the electrical connection according to the invention despite this ability to rotate.

The continuation part of the plug pin can have a tubular or sleeve-shaped construction and the wiping contact arranged on the sleeve part that is fixed relative thereto can apply a force on the outside of this tubular continuation part. It is favorable when the fixed sleeve part encompasses the plug pin or its continuation part on the outside and the wiping contact is arranged in the radial direction between these two parts.

The electrical conductor leading from the metal housing and/or the holder of the helical electrode that is adjustable in the axial direction to the sleeve part—with which the long-lasting electrical connection of the metal housing to the helical coil is established via the wiping contact—can be an additional coil, which is connected or fused in an electrically conductive way to the metal housing holding the helical coil and/or the holder. Such an additional coil can effectively bridge the distance between the distal metal housing and the wiping contact arranged in the proximal region of the pacemaker electrode and can leave the flexibility and elasticity of the pacemaker electrode unaffected and unimpaired due to its coil shape. Above all, any curvature of the pacemaker electrode can be suitably matched by this additional coil, without obstructing any possibly necessary curvature of the pacemaker electrode.

The additional coil can be connected or fused electrically at its proximal end to the fixed sleeve part. Thus, a good connection to the wiping contact is established.

Between the rotating feed line coil and the fixed additional coil, a hose-like or coil-shaped guide can be provided. This can guide, above all, the additional coil and, in particular, can support and stabilize from the inside out, so that it maintains its shape and position even for movements of the pacemaker electrode. Optionally, this guide can also have an insulation function or can be—additional—insulation.

A secure and good holding of the end of the additional coil can be achieved when the metal housing containing the helical coil that can be extended in the axial direction and its holder has a socket-like or hollow projection, which is directed toward the proximal end of the pacemaker electrode and to which the additional coil is attached or fused in an electrically conductive way, preferably on the outside. This projection can be hollow, so that the feed line coil can engage in this projection and can be connected to the holder of the helical coil or to this helical coil itself. Simultaneously, a good and stable attachment is produced for the additional coil.

The wiping contact can be formed by connecting pieces, which extend in the axial direction and which are curved inward and which contact the counter piece with tension and friction and which are arranged one next to the other in the peripheral direction of a surrounding circle and whose cross sections form a circle or polygon that is broken in the peripheral direction. Because the wiping contact engages the rotating plug pin or its projection part from the outside, such connecting pieces that are curved inward in the radial direction or arc-shaped are especially well suited as friction or wiping contacts. Here, these can be connected to each other integrally but are separated from each other by axial or optionally also approximately diagonal slots.

The non-rotating, electrically conductive sleeve part can be connected to a handle piece for the plug pin and can encompass concentrically, within the handle piece, a continuation of the plug pin, which projects at the proximal end opposite the handle piece in the axial direction and which can rotate relative to this together with the feed line coil.

The handle piece is here the protective sleeve known in such plugs for pacemaker electrodes, opposite which the plug pin projects in the distal direction. The mentioned continuation can be arranged within this protective sleeve or within this handle piece and here also within the fixed sleeve part in the radial direction having or supporting the wiping contact, in order to be able to accommodate the wiping contact according to the invention in the region of the distal end of the pacemaker electrode, by which the long-lasting electrical connection of the metal housing to the helical coil is realized.

Above all, for the combination of individual or several of the features and measures described above, a pacemaker electrode is developed, in which, despite a certain movement of the helical coil relative to the metal housing containing it for occasional contacts of these two parts, no electrical voltage pulses are generated, because the two parts are continuously connected to each other electrically by the additional coil.

### BRIEF DESCRIPTION OF THE SEVERAL VIEWS OF THE DRAWINGS

Below, an embodiment of the invention will be described in more detail with reference to the drawing. Shown in schematized representation are:

FIG. 1 is a longitudinal section view of a proximal end of the pacemaker electrode according to the invention with a plug pin, which projects in the proximal direction out of a handle piece of a plug and which is hollow on the inside and which extends within the handle piece through a continuation part in the distal direction up to a feed line coil, wherein the continuation part is enclosed on the outside by a sleeve part, which applies a force on the continuation part with a wiping contact and which is connected in an electrically conductive way to an additional coil, which leads to the distal end of the pacemaker electrode,

FIG. 2 is a section view of the distal end of the pacemaker electrode, in which the additional coil is connected in an electrically conductive way to the metal housing, which contains the helical coil or its holder, and

FIG. 3 is an enlarged scale view showing the construction of the wiping contact for the electrical connection of the additional coil to the plug pin and thus to the feed line coil.

### DETAILED DESCRIPTION OF THE PREFERRED EMBODIMENTS

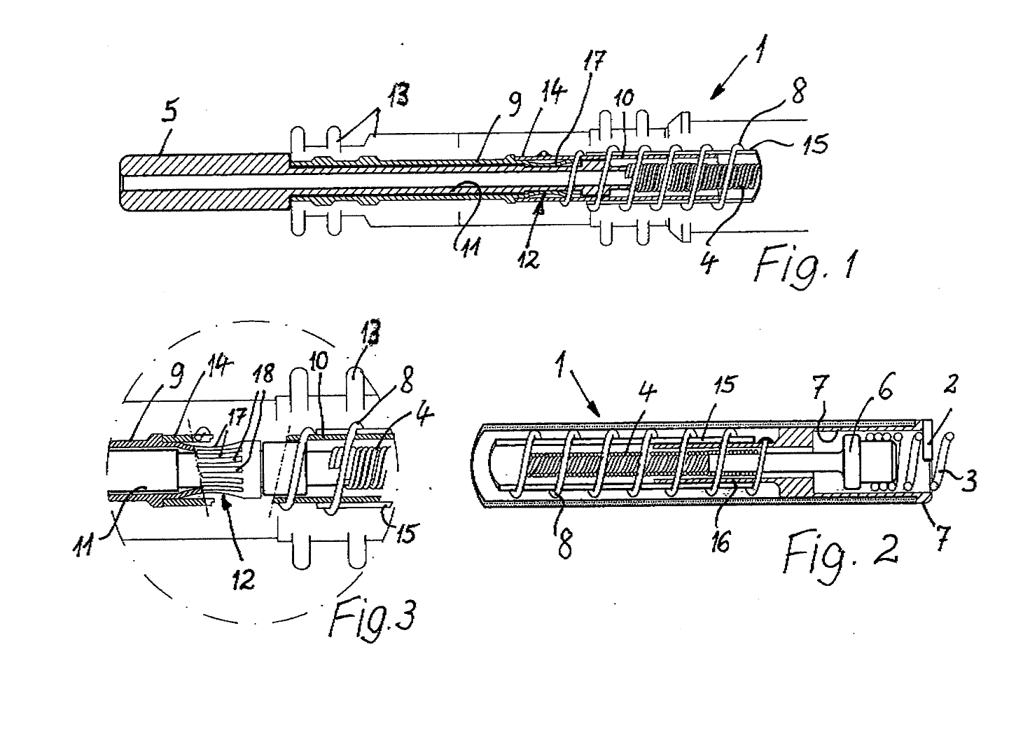

A pacemaker electrode, which is designated as a whole with 1, is shown with its essential parts in FIGS. 1 and 2, wherein these parts shown in FIGS. 1 and 2 are connected in a way that is not shown by a correspondingly long intermediate piece. The pacemaker electrode includes a helical coil 3, which is retracted during its implantation and which can be extended in the axial direction through rotation relative to an engagement part 2 extending between its windings from the retracted position into an active holding position for engaging in the heart tissue and which is connected in an electrically conductive way by an electrically conductive feed line coil 4 to a plug pin 5 that can be introduced into a pacemaker and that has, in the illustrated embodiment, an inner longitudinal cavity.

The helical coil 3 that can be moved by the mentioned rotation in the axial direction into its position of use is here arranged at the distal end of the pacemaker electrode 1 shown in FIG. 2 and held by a holder 6 in a housing 7, which is made from metal and which is also named “metal housing 7” below.

This metal housing 7 can be used as a cathode for measuring the stimulus threshold on the heart for a helical coil 3 still located in its retracted position, whereby the surgeon can also simultaneously recognize that the end face of this metal housing 7 is in contact with the heart tissue, in which the helical coil 3 is then to be screwed in through rotation.

So that no electrical voltage pulses are produced due to contact between the helical coil 3 or its holder 6 and the metal housing 7, a long-lasting electrical connection of the metal housing 7 to the helical coil 3 described in more detail below is provided. If an additional direct contact between the helical coil 3 and the metal housing 7 is generated, then this cannot lead to a voltage pulse or current pulse, because both parts are permanently connected to each other in an electrically conducting way.

For this electrical connection, an electrical conductor 8 is provided, which is connected to the metal housing 7 and/or to the holder 6 of the helical coil 3 that can be adjusted in the axial direction and that is in electrical contact with this metal housing. This electrical conductor 8 leads from the metal housing 7 and/or the holder 6 to a sleeve part 9 still to be explained, and is here, in the preferred embodiment, an additional coil, which is thus also designated below as “additional coil 8” and which is connected to the metal housing 7 holding the helical coil 3 and/or the holder 6 in an electrically conductive way, in the embodiment fused in a way still to be described.

The helical conductor 8 here leads along the feed line coil 4 on its outside to a proximal sleeve-shaped part or sleeve part 10, relative to which the feed line coil 4 and a continuation part 11 of the feed line coil 4 can be rotated, wherein the sleeve parts 9 and 10 are connected in an electrically conductive way and could possibly be integrated.

In FIG. 1 and FIG. 3, one sees that the fixed sleeve part and the rotating continuation part 11 are connected in an electrically conductive way by a wiping contact 12, so that then there is also the long-lasting electrical connection of the metal housing 7 with the helical coil 3.

The continuation part 11 is here connected to the plug pin 5 and also, as already mentioned, to the feed line coil 4, and the wiping contact 12 is provided on the concentrically arranged, non-rotating sleeve part 9, which concentrically encloses, on the outside, the sleeve-shaped continuation part 11 in the region of the wiping contact. The continuation part 11 of the plug pin 5 here has a tubular or sleeve-shaped construction and the wiping contact 12 arranged on the sleeve part 9 that is fixed relative thereto applies a force on the outside of this continuation part 11, as shown in FIG. 1.

The additional coil 8 that is essential for the long-lasting electrical connection of the metal housing 7 with the helical coil 3 is connected in an electrically conductive way at its proximal end by the sleeve part 10 with the fixed sleeve part 9. It is fused thereto with a sleeve piece 14 continuing the sleeve part 10 and contacting this sleeve part 9, but could also be fused directly to the sleeve part 9.

Between the rotating feed line coil 4 and the fixed additional coil 8, in FIGS. 1 and 2 one can see parts of a hose-like or coil-shaped guide 15, which extends across the entire pacemaker electrode and by which the additional coil 8 is stabilized and held and fixed in its preferred coil shape, without negatively affecting its flexibility when the pacemaker electrode bends, because this guide 15 is also correspondingly flexible.

In FIG. 2, the metal housing 7 is shown containing the helical coil 3 that can be extended in the axial direction and its holder 6 has a protective sleeve-shaped projection 16, which is directed toward the proximal end of the pacemaker electrode 1 and at which the electrical conductor 8 provided according to the invention, that is, the additional coil 8, attaches in an electrically conductive way and is fused on the outside in the embodiment.

In the common view of FIGS. 1 and 3, it becomes clear that the wiping contact designated as a whole with 12 is formed by connecting pieces 17, which extend approximately in the axial direction and which are curved inwardly and which contact with tension and friction a counter piece, in the preferred embodiment the continuation part 11 and which are arranged one next to the other in the peripheral direction of a surrounding circle and whose cross sections form a circle broken in the peripheral direction or, for connecting pieces provided with a straight-lined cross section, a corresponding polygon. Above all, in FIG. 3 it can be seen that between these connecting pieces 17, slots 18 are provided which extend across the longitudinal region of the wiping contact 12 and which then end, in order to allow the corresponding rotation of the wiping contact.

The non-rotating, electrically conductive sleeve part 9 is connected in the embodiment to a handle piece 13 for the plug pin 5, wherein this handle piece 13 is formed by a typical protective sleeve, which can be gripped, in order to introduce the plug pin 5 into a pacemaker. Within this handle piece 13, the sleeve part 9 comprises the continuation part 11 connected to the plug pin 3 concentrically, but optionally with some play or distance, in order to allow the relative rotation of the plug pin 3 and this continuation part 11, with which the feed line coil 4 connected to this continuation part 11 according to FIG. 1 can be rotated, in order to rotate the helical coil 3 on its side. In a known manner, the plug pin 3 projects at the proximal end in the axial direction relative to this handle piece 13, in order to allow the described relative rotation of the plug pin 3 together with the feed line coil 4 relative to this handle piece 13.

The pacemaker electrode 1 has a helical coil 3 arranged in a metal housing 7. So that no undesired electrical voltage pulse can occur, if there is mutual contact between the helical coil 3 and metal housing 7, a permanent electrical connection of the metal housing 7 to the helical coil 3 is provided. This electrical connection is realized via an electrical conductor 8, which is advantageously constructed as an additional coil and which is connected directly or indirectly to a part, which is permanently connected in an electrically conductive way to the rotating plug pin 4 and the feed line coil 4 connected to this plug pin and leading to the helical coil 3 via a wiping contact 12.

1. Pacemaker electrode (1) comprising a helical coil (3), which is in a retracted position during its implantation and which can extend in an axial direction through rotation from the retracted position into an active holding position for engaging in heart tissue and which is connected in an electrically conductive way to a plug pin (5) that can be introduced into a pacemaker via an electrically conductive feed line coil (4), the helical coil (3) that can move in the axial direction into the active holding position is arranged in a distal end of the pacemaker electrode (1) and is held by a holder (6) in a housing (7), which is made from metal and which is constructed as a cathode for measuring a stimulus threshold on the heart for the helical coil (3) still in the retracted position, a permanent electrical connection of the metal housing (7) is provided with the helical coil (3) of the pacemaker electrode (1), at least one of the metal housing (7) or the holder (6) of the helical coil (3) that can be adjusted in the axial direction are connected to an electrical conductor (8), which leads along the feed line coil (4) to a proximal, sleeve-shaped part (10), relative to which at least one of the feed line coil (4), a continuation part (11) of the plug pin (5), or the feed line coil (4) can rotate, and the fixed sleeve part (10) and at least one of the rotating continuation part (11) or the rotating feed line coil (4) are connected in an electrically conductive way by a wiping contact (12).

2. Pacemaker electrode according to claim 1, wherein the plug pin (5) or the continuation part (11) connected to the plug pin (5) is connected to the feed line coil (4) and the wiping contact (12) is provided on a non-rotating sleeve part (9) arranged concentric thereto.

3. Pacemaker electrode according to claim 2, wherein the continuation part (11) of the plug pin (5) has a tubular or sleeve-shaped construction and the wiping contact (12) arranged on the sleeve part (9) that is fixed thereto applies a force on an outside of the continuation part (11).

4. Pacemaker electrode according to claim 2, wherein the electrical conductor (8) leading from the at least one of the metal housing (7) or the holder (6) of the helical electrode that can be adjusted in the axial direction to the sleeve part (9) is an additional coil, which is connected or fused in an electrically conductive way to the at least one of metal housing (7) or the holder (6).

5. Pacemaker electrode according to claim 4, wherein the additional coil (8) is connected or fused electrically to the fixed sleeve part (9) on a proximal end thereof.

6. Pacemaker electrode according to claim 4, wherein a hose-like or coil-shaped guide (15) is provided between the rotating feed line coil (4) and the fixed additional coil (8).

7. Pacemaker electrode according to claim 4, wherein the metal housing (7) containing the helical coil (3) that can be extended in the axial direction and the holder (6) has a nozzle-shaped projection (16), which is directed toward a proximal end of the pacemaker electrode (1) and on which the additional coil (8), attaches or is fused in an electrically conductive way.

8. Pacemaker electrode according to claim 1, wherein the wiping contact (12) is formed by connecting pieces (17), which extend approximately in an axial direction and which are curved inward and which contact a counter piece with tension and friction and which are arranged one next to the other in a peripheral direction of a surrounding circle and have cross sections that form a circle that is broken in the peripheral direction.

9. Pacemaker electrode according to claim 2, wherein the non-rotating, electrically conductive sleeve part (9) is connected to a handle piece (13) for the plug pin (5) and, within the handle piece (13), concentrically surrounds the continuation part (11) of the plug pin (3), which projects in the axial direction relative to the handle piece (13) at the proximal end and can rotate relative thereto together with the feed line coil (4).
